(19) United States
(12) Patent Application Publication
(10) Pub. No.: US 2009/0177050 A1
(43) Pub. Date: Jul. 9, 2009

(54) INTEGRATED MEDICAL IMAGING SYSTEMS

(75) Inventors: David M. Griffiths, Pittsburgh, PA (US); David A. Mishler, Slippery Rock, PA (US); Arthur E. Uber III, Pittsburgh, PA (US)

Correspondence Address: GREGORY L BRADLEY;MEDRAD INC, ONE MEDRAD DRIVE, INDIANOLA, PA, 15051, US

(73) Assignee: MEDRAD, INC., Indianola, PA (US)

(21) Appl. No.: 12/374,016

(22) PCT Filed: Jul. 17, 2007
(86) PCT No.: PCT/US07/73673

§ 371 Date: Jan. 15, 2009

Related U.S. Application Data
(60) Provisional application No. 60/807,532, filed on Jul. 17, 2006.

(51) Int. Cl. A61B 5/05; A61B 5/00; A61M 5/145

(52) U.S. Cl. 600/301; 600/407; 604/150

(57) ABSTRACT

An image acquisition system operable to obtain an image of at least a portion of a body, includes an imaging system including at least one energy sensor to measure energy from the body, an image creating system adapted to create an image based at least in part from a signal from the at least one energy sensor, an image display in operative connection with the image creating system and a user interface in operative connection with the image creating system. The image acquisition system further includes a fluid injector system including at least one source of a first fluid, a pressurizing system in operative connection with the source of the first fluid, and a user interface in operative connection with the pressurizing system. The imaging system and the injector system are operatively integrated in at least two of the following and/or other aspects: physical connection, data input via at least one common user interface, displaying of information via at least one common display, electrical connection to at least one common power conditioning system, receipt of common data from at least one patient physiological sensor, at least one common communication port to at least one information system, and a common control system (including any common portion of a control system).

### BACKGROUND OF THE INVENTION

The present invention relates generally to integrated medical imaging systems and, particularly, to integrated medical imaging systems in which one or more components of an imaging system are integrated with one or more components of a fluid injector system.

The following information is provided to assist the reader to understand the invention disclosed below and the environment in which it will typically be used. The terms used herein are not intended to be limited to any particular narrow interpretation unless clearly stated otherwise in this document. References set forth herein may facilitate understanding of the present invention or the background of the present invention. The disclosures of all references cited herein are incorporated by reference.

Angiographic injectors for power injecting imaging contrast into the blood vessel were first developed in the 1960's. They were used in conjunction with X-ray imaging equipment to help with medical diagnosis of circulatory system conditions. Since that time, contrast injectors have been used or contemplated for use in relation to diagnosis conditions in many organs using many types of imaging energy, for example, X-Ray fluoroscopy, CT imaging (computerized tomography), MRI (magnetic resonance imaging), ultrasound, nuclear medicine (NM), PET (Positron Emission Tomography), SPECT (Single Photon Emission Computed Tomography) and visible and infrared light imaging.

The two devices, a fluid injector and an imaging system (imagers) involve some overlapping technologies or disciplines and some very different technologies or disciplines. Fluid injectors generally involve sophisticated electromechanical servo control and assemblies, feedback, and pressure limitation to provide precise steady or time varying injections and well as human factor and safety systems to prevent over injection and reduce the likelihood of air injection. There is normally a controlled progression of states from idle (program and/or fill) to armed (ready to inject) to injecting. Injector systems generally involve some reusable electromechanical and electronic components as well as disposable sterile fluid path elements and systems components. Imaging systems or devices are relatively higher cost capital pieces of equipment. There are commonly no disposable components in imaging systems except for contamination covers in the case of ultrasound probes or other patient contacting surfaces. Technologies generally used by imagers include power conversion from line power to the imaging energy, sensitive sensors and signal amplification to detect the imaging energy, and image reconstruction algorithms and software to create a human understandable image. Some imagers also include electro mechanical control systems. In the case of nuclear medicine imagers, including PET and SPECT, the imaging contrast or imaging agent itself provides the imaging energy. It contains radioactive atoms which decay. The decay energy, most commonly ultimately in the form of a photon or gamma ray, leaves the body and is detected or measured by the sensitive sensor.

Technologies common to both injector systems and imaging systems include, for example, computerized user interfaces, error checking, mathematics and computations for data manipulation or algorithm implementation, computers or microcomputers, motion or position control, line power conditioning or conversion, shielding, environmental control, and data communications to external devices.

The above and other differences between fluid injector devices or systems and imaging devices or systems has lead to physical and business separation of fluid injectors and imaging devices, which are commonly designed and manufactured by different companies or different organizations within the same company. This separation has also limited the interaction between fluid injectors and imaging systems.

Another factor that has maintained the separation of imagers and injectors is the very different regulatory risks associated with the two products. While both are in the broad Class II classification in the FDA regulatory structure, the most serious hazard with imagers is overexposure of the patient to the imaging energy, which, while not insignificant, is rarely immediately fatal. Thus, companies that design and manufacture one product and are familiar with the regulatory requirements and approval processes associated with that product, can be reluctant to add the regulatory hurdles and risk of the other product.

Interaction or cooperation between current injectors and imagers includes timing coordination between the injection of contrast and image acquisition, which has normally been done manually by the operator pushing a button on the injector and the imagers at the proper times, or through one device triggering the other by means of a simple time synchronization interface after a user selected delay.

U.S. Pat. No. 5,840,026 describes a closer cooperation between the injector and the imager. Feedback from the image or a sensor is used to determine and/or adjust the injection to achieve an improved image. The adjustment can include the doctor or operator in the feedback loop, or can be automatic. In U.S. Pat. No. 6,397,098 B1 a system is described that uses various hardware configurations and communications protocol to unidirectionally or bidirectionally communicate a wide array of injection and/or image related data to improve the cooperation between the systems. US Published Patent Application No. 2005/0203389 A1 describes a method, a system, and an apparatus that allow an operator to control independent injectors and imagers from a common console.

Having independent injector and imaging systems has several physical drawbacks. Commonly an injector is mounted on a pedestal with wheels, as shown in FIG. 1, which is taken from US Published Patent Application No. 2004/0199076 A1. The pedestal commonly has a bulky cable that connects it to the user interface and other electronics. This is difficult to move around the room and can get into the way of patient or operator access to the imager.

An alternative to a pedestal is to mount the fluid delivery components of the injector (the injector head) on an overhead counterpoise system (OCS) attached to the ceiling of the room. This mounting system eliminates the cables on the floor and has the benefit that it can be easily pushed up and out of the way. One problem is that it is usually difficult to move the injector head around to both sides of a CT gantry. Or, as imager components move, there is the likelihood that they would bump into the injector OCS. For example, this generally prevents the use of an OCS in an X-ray fluoroscopy suite. The OCS also has difficulty accommodating a situation where the weight changes significantly over time. In some embodiments of fluid delivery systems, for example, as described in U.S. Pat. No. 5,840,026, large volumes of liquid can be initially installed in the injector and then delivered to a sequence of patients over time. As the fluid is delivered, the weight decreases, and the OCS can have a tendency to rise.

In angiography the injector head is sometimes attached to the patient table. As the table is moved to change the region of the patient that is being viewed by the imager, the relative position between the injector head and the patient does not change. This minimizes the chance that the tubing delivering fluid from the injector head to the patient will place stress on the patient.

The injector user interface is commonly situated on a desk or counter surface in the control room next to the monitor or monitors and keyboard that commonly constitute the imager user interface. This clutters the operators work surface and requires them to move back and forth between two input devices. As sophistication of cooperation between the injector and the imager increases, this becomes more of a drawback.

As sophisticated interaction schemes develop, partitioning of function and the speed of data transmission and processing become a challenge. Current injector and imager interaction is primarily limited to start synchronization, done currently by means of settable delays and optionally a switch closure signal. In the case of a settable delay the device starts operating a certain amount of time after the start button is depressed based upon it's control program. Since it takes some time for the injected fluid to travel in the body from the injection site to the imaging region of interest, the injector usually starts first. When the injection has progressed sufficiently or is completed, the injector closes a relay that sends a signal to the imager. The imager then starts acquiring images after the delay of a certain amount of time based upon it's control program. A common alternative involves placing the injector and imager user interfaces in close proximity on a counter, setting the imager delay to a defined amount of time, and the operator pressing both start buttons simultaneously. This causes the imager to start imaging the defined amount of time from that time when the user depressed both start buttons. A less common alternative is to have the imager trigger the start of the injector through a relay closure and then acquire images after the defined amount of time.

Given recent advances in computers and electronics, information communications methods have been changing rapidly. Imaging systems, image display workstations, and picture archiving and communications systems (PACS) from different manufacturer can communicate information and interact to a significant extent through standards such as DICOM (Digital Imaging and Communications In Medicine) published by NEMA (National Electrical Manufacturers Association) http://medical.nema.org/.

Additionally, the Integrating the Healthcare Enterprise (IHE) initiative is a project designed to advance the state of data integration in healthcare. Sponsored by the Radiological Society of North America (RSNA) and the Healthcare Information and Management Systems Society (HIMSS), the IHE brings together medical professionals and the healthcare information and imaging systems industry to agree upon, document and demonstrate standards-based methods of sharing information in support of optimal patient care.

A medical interface bus standard, IEEE1073/MIB, exists to facilitate communications among various medical devices.

Further, a consortium of injector and imager companies is working on a communications standard for communications between injectors and imagers based upon the CAN Bus

It is desirable to develop integrated medical imaging systems in which one or more components of an imaging system are integrated with one or more components of a fluid injector system.

### SUMMARY OF THE INVENTION

In one aspect, the present invention provides an image acquisition system operable to obtain an image of at least a portion of a body. The image acquisition system includes an imaging system including at least one energy sensor to measure energy from the body, an image creating system adapted to create an image based at least in part from a signal from the at least one energy sensor, an image display in operative connection with the image creating system and a user interface in operative connection with the image creating system. The image acquisition system further includes a fluid injector system including at least one source of a first fluid, a pressurizing system in operative connection with the source of the first fluid, and a user interface in operative connection with the pressurizing system. The imaging acquisition system can further include a communication system placing the imaging system and the fluid injector system in communicative connection. The imaging system and the injector system are further operatively integrated in addition to the communication system. The imaging system can further include at least one energy source to transmit energy into the body.

In several embodiments, at least one component of the imaging system is physically connected to at least one component the injector system. At least a portion of the injector system can, for example, be housed within a housing of the imaging system. At least a portion of a housing of the injector can be attached to the imaging system. A portion of the injector housing can, for example, be attached to a support for the image acquisition system. At least one fluid path in operative connection with the injection system can be in operative connection with the imaging system. At least one fluid reservoir in operative connection with the injection system can be in operative connection with the imaging system. Likewise, at least one fluid heating system in operative connection with a fluid path of the injection system can be in operative connection with the imaging system. The imaging system can include at least one supply compartment adapted to house supplies for the injector system.

The imaging system and the injector system can also be in electrical connection with a common power conditioning system.

The imaging system and the injector system can be adapted to receive common data from at least one patient physiological sensor. The patient physiological sensor can, for example, be an ECG sensor, a respiration sensor, a blood oxygen sensor, or a blood pressure sensor.

The imaging system and the injector system can share a common control system (including any portion thereof). At least a portion of the control software for the imaging system and the injector system can, for example be integrated or distributed throughout the common system's hardware architecture. The imaging system and the injector system can share at least one common state machine state (and/or at least one common transition between machine states). The imaging system and the injector system can integrate imaging parameter and fluid delivery protocols. The imaging system and the injector system can integrate user preferences. The imaging system and the injector system can share patient information. The imaging system and the injector system can share usage data.

The imaging system and the injector system can share at least one common computer component. For example, the imaging system and the injector system can share at least one common computer memory. The imaging system and the injector system can share at least one common computer processor. The imaging system and the injector system share at least one common data communication bus (for example, a PCI or other bus as known in the computer arts).

The imaging system and the injector system can share at least one common safety check system. The imaging system and the injector system can share at least one common user interface. The imaging system and the injector system share at least one common display. The imaging system and the injector system can share at least one common communication port to at least one other information system. The information system can, for example, be a hospital information system.

In another aspect, the present invention provides an image acquisition system operable to obtain an image of at least a portion of a body, including an imaging system including at least one energy sensor to measure energy from the body, an image creating system adapted to create an image based at least in part from a signal from the at least one energy sensor, an image display in operative connection with the image creating system and a user interface in operative connection with the image creating system. The image acquisition system further includes a fluid injector system including at least one source of a first fluid, a pressurizing system in operative connection with the source of the first fluid, and a user interface in operative connection with the pressurizing system. The imaging system and the injector system are operatively integrated in at least one aspect other than communication of data between the imaging system and the injector system.

In another aspect, the present invention provide an image acquisition system operable to obtain an image of at least a portion of a body, including an imaging system including at least one energy sensor to measure energy from the body, an image creating system adapted to create an image based at least in part from a signal from the at least one energy sensor, an image display in operative connection with the image creating system and a user interface in operative connection with the image creating system. The image acquisition system further includes a fluid injector system including at least one source of a first fluid, a pressurizing system in operative connection with the source of the first fluid, and a user interface in operative connection with the pressurizing system. The imaging system and the injector system are operatively integrated in at least two of the following (and/or other) aspects: physical connection, data input via at least one common user interface, displaying of information via at least one common display, electrical connection to at least one common power conditioning system, receipt of common data from at least one patient physiological sensor; at least one common communication port to at least one information system, and a common control system (including any common portion of a control system).

In a further aspect, the present invention provides an imaging system including at least one energy sensor to measure energy from the body, an image creating system adapted to create an image based at least in part from a signal from the at least one energy sensor, an image display in operative connection with the image creating system and a user interface in operative connection with the image creating system. The imaging system is adapted to be integrated with a fluid injector system in at least two of the following (and/or other) aspects: physical connection, data input via at least one common user interface, displaying of information via at least one common display, electrical connection to at least one common power conditioning system, receipt of common data from at least one patient physiological sensor; at least one common communication port to at least one information system, and a common control system.

In still a further aspect, the present invention provides a fluid injector system including at least one source of a first fluid, a pressurizing system in operative connection with the source of the first fluid, and a user interface in operative connection with the pressurizing system. The injector system is adapted to be integrated with an imaging system in at least two of the following (and/or other) aspects: physical connection, data input via at least one common user interface, displaying of information via at least one common display, electrical connection to at least one common power conditioning system, receipt of common data from at least one patient physiological sensor; at least one common communication port to at least one information system, and a common control system.

The present invention also provide method of fabricating the systems of the present invention as well as method of performing imaging procedure using the systems of the present invention.

In several embodiments, the devices, systems and methods of the present invention go beyond the ever expanding communications, control, and processing capacity of the computer and electronic disciplines to facilitate cooperation between independent devices. The devices, systems and methods of the present invention more fully integrate imagers and injectors along multiple system aspects or dimensions, providing significant benefits to the patients, operators, doctors, and manufacturers through physical, informational, and/or operational integration to increase efficiency and/or capability.

The present invention, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a prior art imaging system and a prior art injector system used in connection with the imaging system.

FIG. 1B illustrates another prior art imaging system and a prior art injector system used in connection with the imaging system.

FIG. 2 illustrates one embodiment of an integrated imaging and injector system of the present invention.

FIG. 3 is a block or functional diagram of prior art imager system and injector system.

FIG. 4a is a block or functional diagram of an embodiment of an integrated imager injector system of the present invention.

FIG. 4b is a block or functional diagram of another embodiment of an integrated imager injector system of the present invention.

FIG. 5 is a state diagram of prior art imager system and injector system.

FIG. 6 is a state diagram of an embodiment of an integrated imager injector system.

FIG. 7a illustrates an embodiment of an integrated imager injector system of the present invention.

FIG. 7b illustrates an embodiment of an integrated imager injector system of the present invention similar to that illustrated in FIG. 7a in which the injector is attached to a front of the imager.

FIG. 8 illustrates another embodiment of an integrated imager injector system of the present invention.

FIG. 9 illustrates another embodiment of an integrated imager injector system of the present invention.

FIG. 10 illustrates another embodiment of an integrated imager injector system of the present invention.

FIG. 11 illustrates an embodiment of an integrated imager injector system of the present invention.

FIG. 12a illustrates an embodiment of an integrated imager injector system of the present invention.

FIG. 12b illustrates an embodiment of an integrated imager injector system of the present invention.

FIG. 13 illustrates an embodiment of a physically separable integrated imager injector system of the present invention.

FIG. 14 illustrates an example of a functional diagram of a partially integrated imager injector system of the present invention.

### DETAILED DESCRIPTION OF THE INVENTION

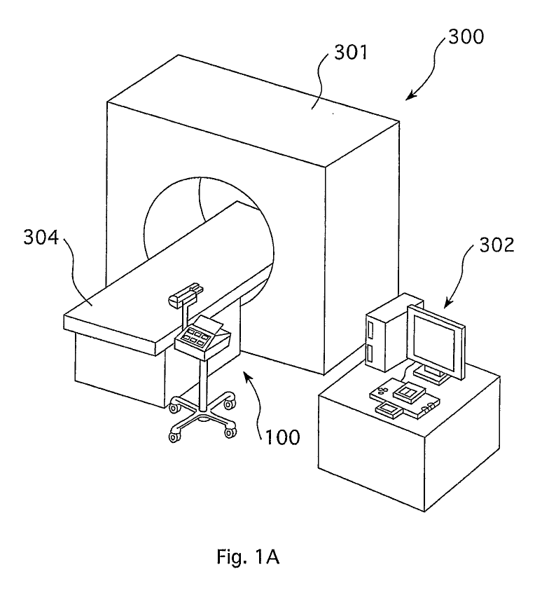

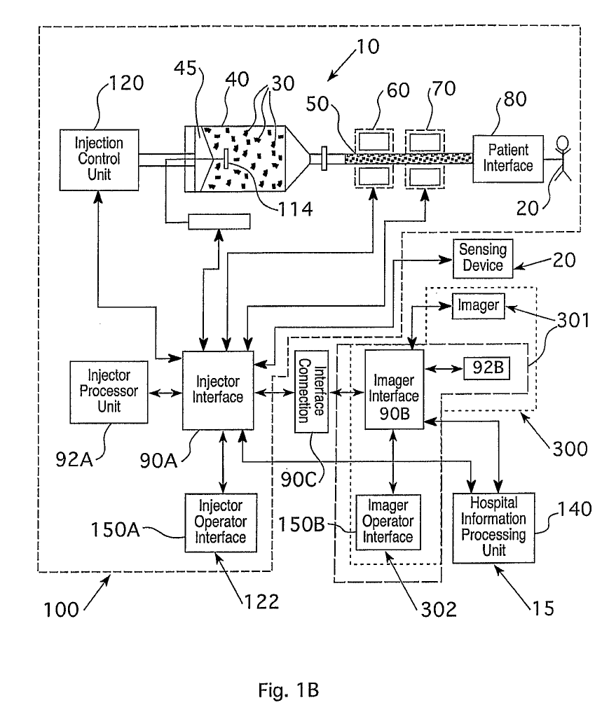

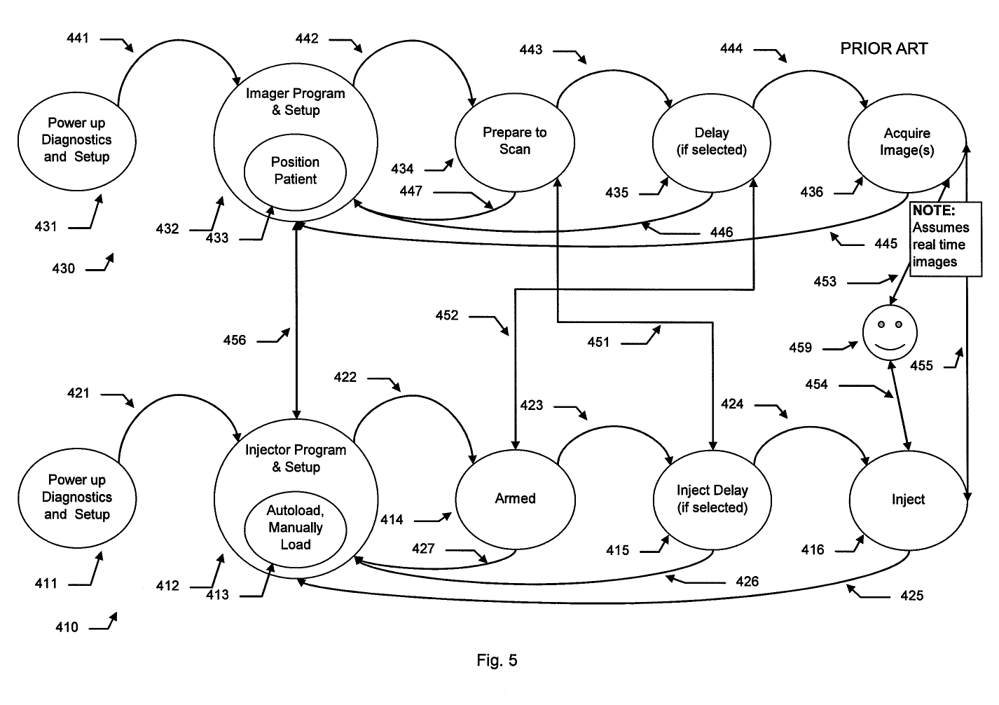

FIG. 1a illustrates a prior art system based on FIG. 5 of Published U.S. Patent Application No. 2004/0199076 A1, illustrating the typical physical and functional separation of an injector 100 from an imager system 300. For ease of understanding, the imager 300 can be considered to be a CT scanner. The image acquisition apparatus 301, commonly called a gantry in CT parlance, contains an X-ray tube that emits X-ray energy as its imaging energy and sensors that measure the X-rays after they have passed through the patient. This information is used by an algorithm, implemented in a computer program in the imager to create an image which can be displayed on the imager user interface 302, commonly called a user console, or sent through the hospital information system or network to other devices, commonly referred to as remote viewing or work stations. The patient is placed on the patient couch, also called a support, positioner, table, or bed 304 and positioned so that the correct region or portion of the body is imaged. The CT scanner is programmed and set up with imager user interface 302 as well. During the scan, progress can usually be monitored on the imager user interface 302.

The separate injector 100 can be moved around with respect to the imager system 300 to facilitate injection of the patient. The injector injects contrast medium that affects the image of the patient, yielding additional diagnostic information. The injector has a user interface 122 that can be used to program the injector and to monitor injection status during an injection. The injector 100 has an injector head 121 that in this example holds two syringes, one for contrast medium and one for saline. Such injectors, control systems therefore and injector protocols used therewith are described, for example, in U.S. Pat. Nos. 6,643,537, 6,339,718, 6,673,033, 6,767,319, 6,958,053 and 5,494,036 and in Published U.S. Patent Application Nos. 2004-0064041, 2005-0113754, the disclosures of which are incorporated herein by reference. The injector head 121 commonly also contains one or motors that are used to pressurize the contrast medium and thus determine and control its flow into the patient. The fluid flows from the injector head 121 to the patient through a fluid path that is not shown. Often there is a remote user interface for the injector, not shown in this figure, that sits on the same work surface as the imager system user interface 302, and in the case of a CT system these are usually outside of a radiation shielded room to minimize the radiation dose to the operator.

For imaging systems, such as ultrasound, where there is no hazard to the operator from the imaging energy, the imaging system user interface 302 is often physically part of the imager 301 that is commonly moved with respect to the patient bed 304. In this case the injector 100 is commonly also moved with respect to the patient bed 304.

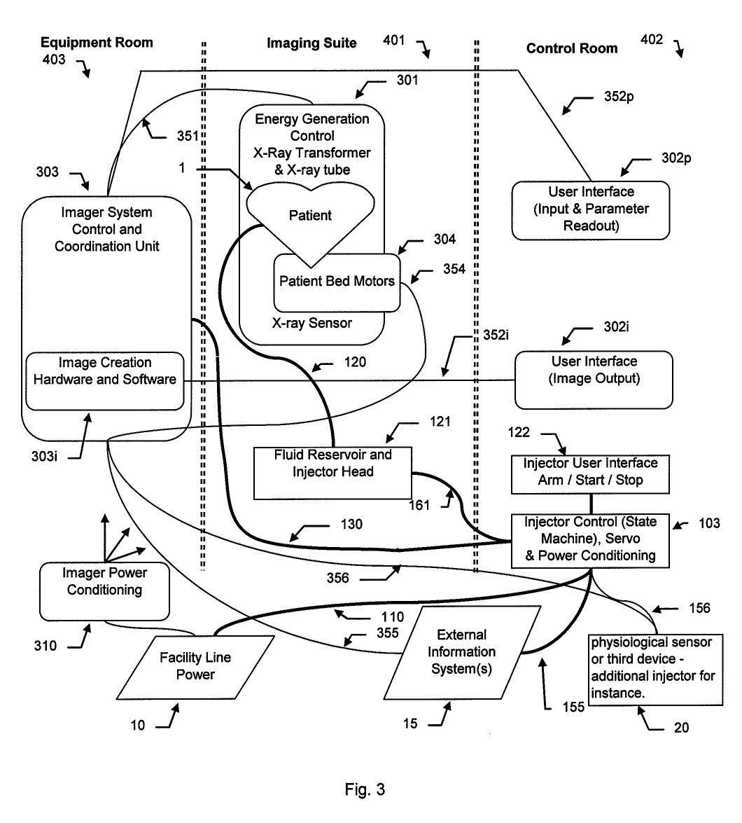

FIG. 1B is based on FIG. 3 of U.S. Pat. No. 6,397,098 B1, the disclosure of which is included herein by reference. U.S. Pat. No. 6,397,098 discloses devices and methods for communicating information between the independent injector 100 and imaging system 300 to promote better cooperation between independent injector 100 and imaging system 300.

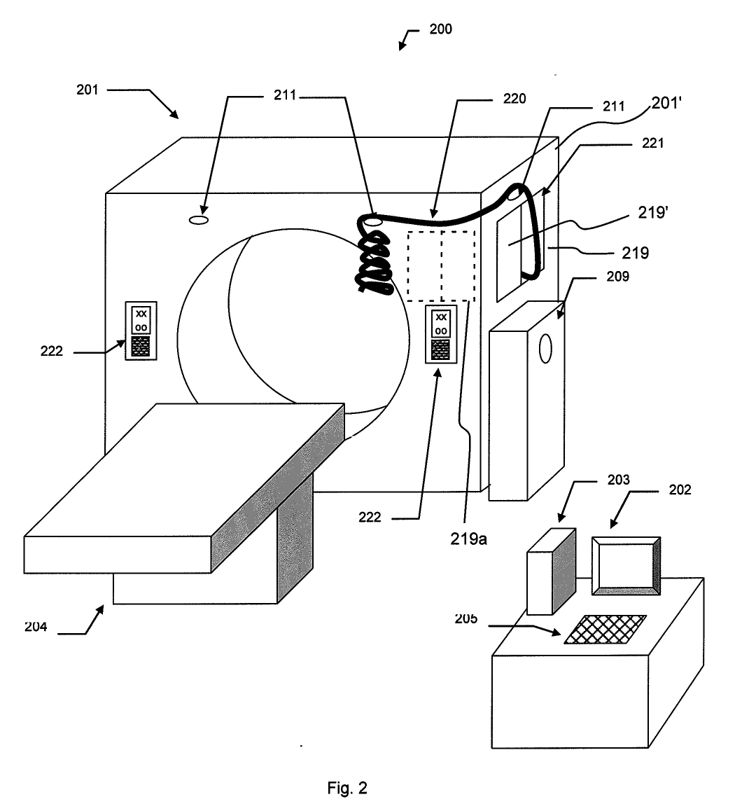

FIG. 2 illustrates an embodiment of the present invention in which there is a single integrated imager injector system (IIIS) 200. The imager and injector functions are integrated to an appropriate extent. This integration has many benefits that are discussed in further detail below. Among these benefits are space savings, material savings, cost savings, time savings, and achievement of imaging protocols and capabilities (and thus diagnostic capabilities) that are not possible or possible only with substantial difficulty using prior art systems.

As an illustrative example, FIG. 2 sets forth an integrated injector CT imager. In this system, the patient lies on the IIIS patient support surface 204 (also called a couch or a bed) and is positioned properly with respect to the IIIS image acquisition apparatus 201. The fluid injector or injector head 221 is incorporated into the image acquisition apparatus 201. In this example, a housing 201′ of image acquisition apparatus 201 includes an injector seating or compartment 219 having doors 219′ that are preferably clear so that the operator can observe the state of the fluid injector 221. The doors, which are optional, can serve to heat and maintain the fluid and aspects of the fluid path near body temperature for patient comfort. In the illustrated embodiment, compartment 219 is illustrated on the side of housing 201′. As illustrated in dashed lines, an injector compartment 219a can be placed at other positions within housing 201′ (for example, on the front thereof) based upon operator ease of use or based upon the existence of available space in the imager. A fluid line 220 goes from the injector 221 to the patient area. Mounting aids 211 attached to housing 201′ help support the fluid lines in a convenient fashion. For CT, there can be mounting aids (not visible, but similar in design and operation to mounting aids 211) on the back side of the image acquisition apparatus 201. as the patient is sometimes positioned head first into the scanner with the patient's arms over the patient's head, and the IV catheter in the patient's arm. To keep the fluid line 220 out of the image, it can be connected to the IV catheter in the patient's arm on the back side of the image acquisition apparatus 201. Optionally, segments of the fluid line 220 can be mounted inside the housing 201′ so that they are less likely to be accidentally bumped or dislodged by a user or patient. This can be especially useful for segments that are used for multiple patients.

The fluid injector 221 could for example be a multipatient system as disclosed in U.S. Pat. Nos. 5,806,519, 5,885,216, 5,843,037, 6,149,627, 6,306,117, 5,739,508, 5,920,054, 5,569,181, 5,840,026, the disclosures of which are included herein by reference. The source of the fluid in this example can, for example, be bags, bottles or other containers.

There are IIIS user interfaces 222 on either side of the patient bed 204. These can, for example, be “simplified” user interfaces which do not include the full functionality of a more complete user interface system associated with the imaging system or the injector 221. Each of user interfaces 222 can, for example, allow position control of the patient couch 204, some function of the fluid injector 221, and tilt or positioning of the image acquisition apparatus 201. The more complete user interface is usually located in the control room and includes, for example, a display 202 and input device 205, which is optionally a keyboard and mouse or trackball. Computer 203 can be in close proximity to the display 202 and input device 205 or be at a reasonable distance, or even be physically dispersed among various system components.

Specifically for CT, two generally identical posts 209, of which one is visible from the perspective of FIG. 1, usually support the image acquisition apparatus 201. Supports 209 allow the image acquisition apparatus 201 to take angled images that are, for example, especially useful in some head imaging protocols. In angiography the patient table commonly moves vertically and generally in one horizontal direction. For MR, the image acquisition apparatus (the magnet and associated coils) is generally immovable and the patient is moved with respect to it. For ultrasound, the image acquisition apparatus is often hand held, so it is readily movable with respect to the patient who generally is not moved. The movement and various positional adjustment of other medical imaging acquisition apparatuses 201 or patients with respect to the apparatuses are well know to those practiced in the medical equipment arts.

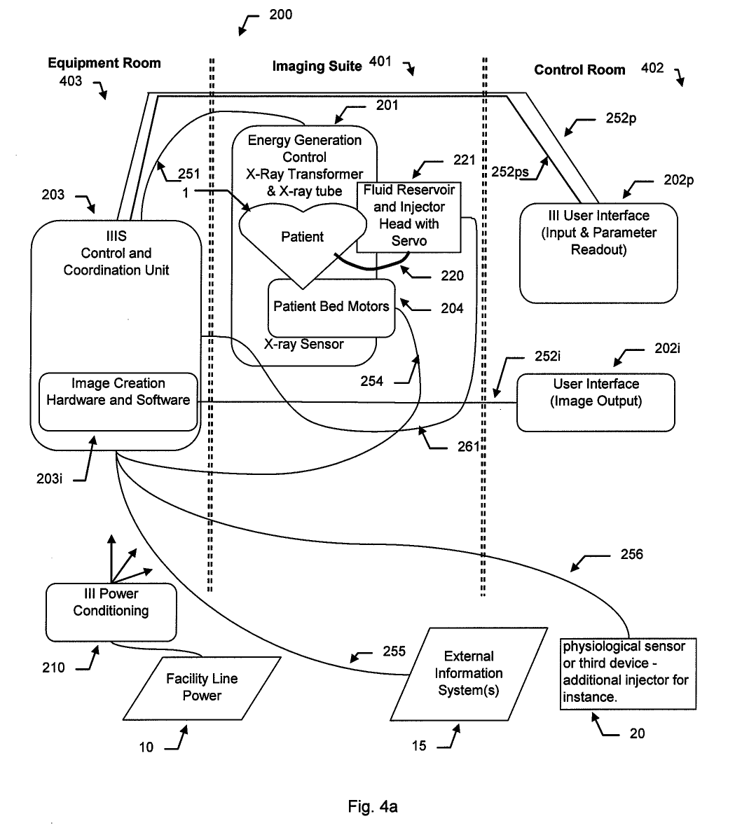

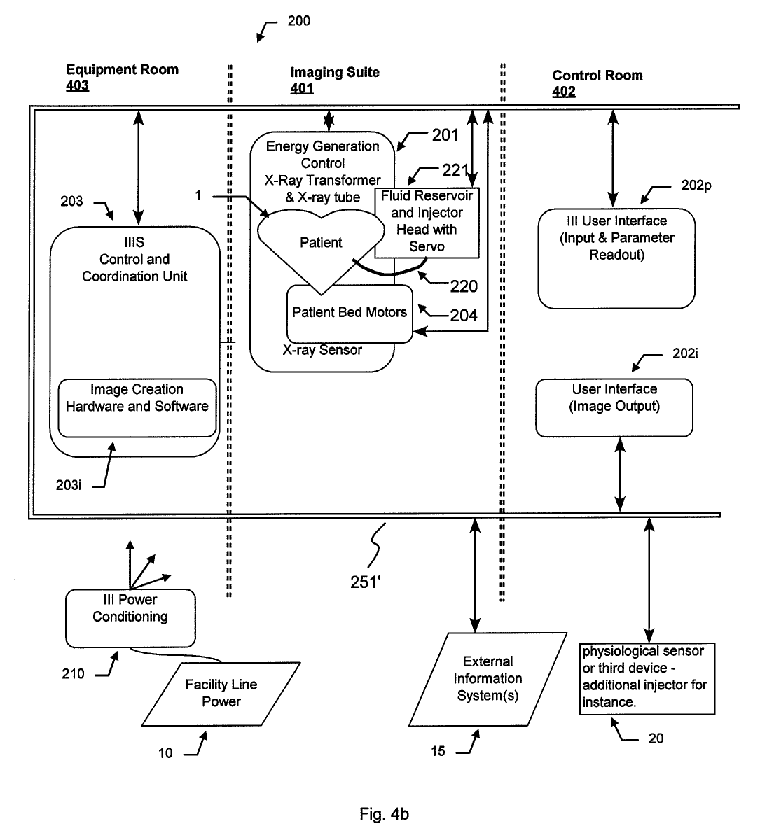

To better understand the level of integration of this invention and the benefits that are possible, a comparison of the block diagram of the prior art, as shown in FIG. 3, will be made with an exemplary block diagram of this invention, as shown in FIGS. 4a and 4b. Each of the figures shows a system distributed between three rooms: an imaging suite 401, a control room 402, and equipment room 403. This is typically the case for CT imaging and almost always the case for MR imaging, which has significant support electronics. However, sometimes the devices normally housed in the equipment room can be dispersed between the imaging suite and the control room. For a simpler imaging modality such as ultrasound, all the components can be, and usually are, housed in the imaging room, and potentially in a single housing 201′.

In each of FIGS. 3, 4a and 4b, there is electrical power 10 provided by the hospital facility. Even if the injector 100, imager 300, or IIIS apparatus 200 is battery powered or solar cell powered, ultimately the power is supplied from the facility either in recharging or replacing the batteries. There is also commonly a network or information system 15 to which the injector 100, imager 300, or IIIS apparatus 200 interfaces. This network 15 can provide communications to systems that, for example, collect patient records, store or archive images, CAD (computer aided diagnosis), collection data or procedures, or are used for patient or healthcare payor billing, inventory control, per use billing by IIIS equipment supplier or owner, and equipment monitoring, servicing, and maintenance. The network 15 can optionally provide communications to other imaging systems that may be in the same imaging room, or in different rooms. Further, there can be other devices generally shown as 20 with which the injector 100, imager 300, or IIIS apparatus 200 interface. These other devices can, for example, include sensors (for example ECG, pulse rate, respiration monitors, blood pressure monitor, EEG, skin galvanic response, pulse oximeter, evoked potentials, video observation of the patient or some aspect of the patient, for example eye motion, etc.) or one or more additional devices such as another fluid delivery device or a respirator.

In the prior art system of FIG. 3, the injector includes an injector head 121 (often with a fluid source or reservoir) that pressurizes the fluid for delivery. It also includes a user interface 122 through which the user specifies the actions of the injector. An injector control 103 interfaces with the user interface 122, the imager 300 via communications line 130, any additional devices 20 via line 156, the external information system 15 via communications path 155, and the source of power via line 110. The line power is generally reduced and rectified to be used by the servo and reduced to 5V or 3.3 V for the processor or logic circuits. The injector control generally includes a state machine and servo control so that the pressure of the fluid is controlled to achieve a flow or delivery characteristic that provides a diagnostic image, often according to a predetermined flow profile. The state machine, servo control, and user interfaces are usually implemented using one or more computers and associated software. As disclosed in U.S. Pat. Nos. 5,840,026 and 6,397,098, improved communications and thence cooperation between imager 300 and the injector 100 result in improved capability for concurrent contrast delivery and image acquisition optimization and improved diagnostic images.

In the prior art system of FIG. 3, the imager 300 (a CT imager, for example) has a sophisticated image acquisition apparatus 301 that includes X-ray generation and sensing devices. The patient is placed in the proper position by a motorized bed or couch 304. There is an imager control and coordination device 303, that usually includes one or more computers, communicates with the image acquisition apparatus 301 via path 351, with the patient bed 304 via path 354, with a control user interface 302p via path 352p and with a image output user interface 302i via path 352i. These two user interfaces may be the same physical device. There may be multiple output user interfaces 302i. For later discussion, it is worth highlighting a specific aspect of the imager control and coordination device 303, the image creation section with hardware and software 303i that creates the images from the energy measurements. The imager control and coordination device 303 also interfaces to the external network or information system 15 via path 355, for example a local area network, intranet, or internet, and additional sensors or devices 20 via path 356. Generally, in CT and MR, the time needed to create an image once the data is acquired is such that the images cannot be created in real time, although the image creation time is becoming increasingly rapid and approaching real time in some instances. In ultrasound and X-ray fluoroscopy, real time images are readily available in real time.

The prior art imager also includes power supply or conditioning circuitry 310 that provides power to all the appropriate imager segments. X-ray tubes require very high voltages. X-ray measurement circuits logic generally operates on 3.3 or 5 volts. The paths from power conditioner 310 are not shown to improve clarity.

FIG. 4a shows an embodiment of the integrated imager injector system 200 of the present invention. The IIIS user interface 202p is used to input control and program information for both the imager and the injector user interface functions. It includes both output/display devices and input devices. The IIIS system control and coordination unit 203 controls both the injector and the imager. It can utilize a simple physical computer or two or more computers in communication through a sufficiently high-speed communications path. Communications path 252i provides data to another device, for example a 3D rendering device or separate image viewing workstation 203i. The IIIS power conditioning 210 supplies power to the IIIS components and subsystems.

There are multiple benefits derived from power supply or conditioning integration 210. For example, if one component or subsystem draws too much current and blows a fuse, the IIIS control and coordination unit 203 can recognize this state and stop the whole procedure, saving the patient unnecessary radiation exposure and/or drug delivery, or simply preventing wasted time. With currently independent injector and imager systems, failure of power to one system or subsystem doesn't generally stop the other. The integrated power conditioning equipment 210 can also be more efficient, converting the AC to various DC or high voltage needs with less redundancies than separate units. And, because it is a single unit, more expensive and effective protection strategies can be economically employed. There is also a user convenience and simplicity benefit and a time savings simply by having one on/off switch control the power to the whole IIIS rather than separate switches for the injector and imager. Power conditioning often involves the generation of excess heat. The integration of the IIIS can also facilitate the heat dissipation and other environmental aspects of the various system aspects. For example, performing the power conditioning remotely from the image sensor and the injector can reduce the heat dissipation capability needed in those two parts.

All interfaces 255 & 256 to external devices can, for example, be controlled by the IIIS system control and coordination unit 203. This integration also facilitates the collection of all patient safety and procedure related information such as radiation dose and/or drug dose for monitoring or statistical analysis. The integration enables one set of data about the injection to be provided to all external systems (for example for patient records, picture archiving, payor billing, purchasing of supplies, per use billing by equipment supplier(s), equipment servicing, operator training etc.) rather than the external system having to try to coordinate and correlate data from separate devices that relate to a single patient's imaging procedure, thereby avoiding the potential for loss of data, data misalignment or duplication, and other data coordination errors inherent in two independent systems sending data to a third system. The physical operation of the mechanical systems to pressurize the fluid 221, position the patient 204, and acquire images 201 operate normally, with some simplifications and the benefits as discussed elsewhere herein.

In FIG. 4b, various communication lines and/or interfaces as set forth in FIG. 4b are replace by a common communication/data bus 251′ (for example, a PCI bus as known in the art). Each of the various system components, as described in connection with FIG. 4a, are placed in communicative connection with data bus 251′.

The block diagrams of FIGS. 3, 4a and 4b are simplified or limited in detail for clarity of explanation of the benefits and embodiments of an integrated imager injector system. The further details of design are well known to those skilled in the art. For example the user interfaces 222 shown in the imaging suite in FIG. 2 are not shown as separate user interfaces in FIGS. 4a and 4b. The communications paths in the embodiment of FIG. 4a can, for example, be conductive wires or cables of various designs, fiber optic conduits, or wireless communication devices, such as RF or infrared. Multiple types of communications paths may be used, even in one system. In addition, the various physical devices and software operating on the devices may be manufactured by various manufactures and integrated into the IIIS by the final manufacturer who supplies the device to the user. From the user's point of view, having one manufacturer responsible for the whole IIIS is beneficial, even if that manufacturer sends various specialized repair personnel to maintain different components or sends defective components to various companies for repair. An integrated system allows the manufacturer to readily perform preventative maintenance and/or (for example, through remote diagnostics) to pinpoint whether a problem is in what might be called the imager subsystems, the injector subsystems, or the common subsystems and thus more accurately diagnose and more quickly repair a problem or problems.

Integration further provides for combined applications/training opportunities. Moreover, purchasing is simplified. For example, in a fully integrated system a single order/purchase can be effected for the entire IIIS system. Branding can, for example, occur under a single name.

FIG. 5 is a simplified state diagram for prior art injector system 100 operating in conjunction with or cooperation with an imager system 300. The injector system state diagram is indicted generally as 410. It includes states for power up diagnostics and setup 411, injector program and setup 412, armed (able to inject) 414, delay 415, and inject 416. When power is first applied to the injector, it starts in the power up and diagnostics mode 411. A number of checks are performed, and if the injector passes, it transitions via path 421 to injector program & setup state 412. In this state the user can program a multi-phase injection with various fluids and flow rates, (including 0 flow for a specific or indeterminate length of time, which is often termed pause or hold). The user can also program delays that relate to the synchronization of the injection and image acquisition as discussed herein. The doctors or the hospitals preferred injection protocols can be stored in memory for easy recall. This standardization also reduces the chance for operator error when entering the various numbers needed to full specify an injection protocol. While in this setup state 412, the user can also cause the movement the various fluid flow pressurizing elements, for example, to fill a syringe or prime a fluid line. In sub state 413, it is up to the user to make sure that there in no risk to the patient from any injector action or motion, either manually controlled or automatic, such as the auto prime function available on the STELLANT® injector manufactured by Medrad, Inc. of Pittsburgh, Pa. Once everything is ready for the injection and the fluid path is properly connected to the patient, the operator goes through a sequence of one or more button pushes and question responses 422 that transitions the injector to the armed state 414. In this state, a trigger from the imager via 452, if they are set up to cooperate in this manner, or from the user can cause the injector to move into the inject delay state 415, if one has been programmed. The injector generally moves to the inject state 416 after the programmed time delay, if there is one, or in the programmed time relation to a signal 451 from the imager. Once injecting fluid, current devices, for example the STELLANT injector, executes the programmed injection to completion unless the user 459 causes a change 454 in the injection. In U.S. Pat. No. 5,840,026, the user 459 can, for example, cause or make changes in the injection based upon the image observed 453. Alternatively, if the injector and imager are set up to cooperate in this way, digital information such as pixel density can be transmitted from the imager 300 to the injector 100 to be used to adjust the injection through communications path 455 if the images can be created in a sufficiently short period of time that the feedback can be effective.

When the injection is completed, the state returns to injector program and setup state 412 via 425. If there is an error detected by the injector or the operator, the injector can abort or stop the injection and quickly change to the injector program & setup state 412 via state transitions 427, 426, or 425.

An imager has a similar state diagram, shown generally as 430. Upon first applying power to the imager, it goes through diagnostics and setup state 431. This may involve warming up components such as X-ray tube filaments. When everything is completed and checks out OK, the imager transitions via 441 to the imager program and setup state 432. Here the parameters for the study are set. It may be possible for the operator to preset parameters and other things while the imager is going through it's power up diagnostics. The many parameters for selected standard scans can be saved in memory for easy recall. This recall also reduces the chance of operator error.

When the operator believes everything is ready and in position, and if the imager confirms that things are in order, the state moves through transition 442 upon direction of the operator to the prepare to scan state 434. Upon a trigger from the injector 451 or an indication from the user, transition 443 moves the state to the optional delay state 435. As with the injector, there may be no delay, or the delay may be a wait for a signal 452 from the injector if they are set up to cooperate in that way. When the delay state criteria is fulfilled or completed, transition 444 takes the imager to the image acquisition state 436. When the image acquisition is complete, the system transitions 445 back to the setup state 432. The system can abort the imaging sequence as a result of system error or user choice in any of these states and return to the setup state via transitions 445, 446, or 447.

As mentioned above, if the imaging system can provide images in a sufficiently rapid time frame, image information can be used to adjust the injection either directly via, for example, communication path 455 or through a human operator 459 via 453 and 454. Alternatively, as discussed in U.S. Pat. No. 6,397,098 the conditions of the injection may be used to modify the parameters of the image acquisition either directly or through the operator as indicated by the upward arrows 455, 454, and 453. Rapid imaging and adjustment can also be beneficial which conducting a biopsy procedure, either manual or automated, which involved sampling some tissue from the patient, commonly via a needle being inserted under image guidance.

U.S. Pat. Nos. 5,840,026 and 6,397,098 also disclose communications 456 between the injector and imager in their respective setup states. Many uses and additional details are provided therein.

In some cases, it is necessary to take what is termed a scout image to determine the exact region of the body to be imaged. In this case, the imager transitions from state 432 through to 436 and then back to 432 with the scout image now being used by the operator or software in setup state 432 to finalize the parameters. In MR, a pre-scan is often done to tune a number of parameters to the particular patient and their characteristics before a diagnostic scan. A three plane localizer imaging sequence is also commonly done to allow the operator to select the specific volume to be imaged.

In some imaging modalities such as CT and MR, the prepare to scan state 434 is very important, because starting a scan accidentally can result in patient harm. In other imaging modalities, such as ultrasound there is no need for a separate prepare to scan state and less of a need for a scan delay state in the current practice. In ultrasound, because the imaging energy is relatively harmless, adjustments and program changes are often made while the imaging energy is being applied and images are being created, especially because the adjustments are made based upon the operator's assessment of the images. Some infrared and visible light systems may be operated similarly.

As is evident from the complexity of even this simplified state diagram, the operator has to provide significant coordination between the two systems. And, there are a number of communication paths that can be problematic or can malfunction for the systems to cooperate in even a slightly more automated manner. For example, the operator has to manually match the injection protocol to the selected scan protocol.

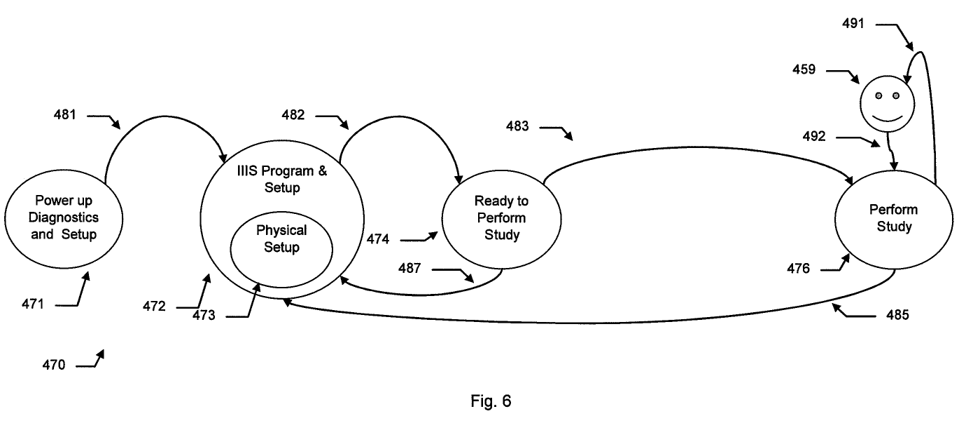

FIG. 6 shows an embodiment of a significantly simplified state diagram of an embodiment of an integrated imager injector system 200 of the present invention. On initial power up, the integrated imager injection system 200 checks all components while in state 471. When this is satisfactorily completed, the system enters the IIIS program and setup state 472 in which the user can prepare and program the total for the desired study. The chance of operator error is reduced because any stored protocols or programs include both scanner and one or more associated fluid delivery protocols, so that the operator cannot mismatch the two programs or protocols as is possible in prior art systems. In this state the operator physically positions the patient, connects the fluid delivery line and does all the other work necessary for the imaging study as indicated by substate 473. When the IIIS system 200 is ready for imaging, the user initiates the change to the perform study state 482. Before transitioning, the system does a prescribed set of check and interactions with the operator, such as checking for air and proper patient positioning. If everything checks out satisfactorily, then it transitions via 482 into the ready to perform study state 474. Because of the integration of the system, there is no need to confirm communications between the different devices. This can, for example, be covered by initial and periodic or continuous internal hardware and software checks and exception handling routines. The operator then triggers the system to transition 483 to perform the study 476. The timing of all phases, both fluid delivery and image acquisition can have been coordinated by the operator either directly or via choices she/he has made. The use of image results to adjust fluid delivery or image acquisition can occur seamlessly, controlled by a single algorithm, optionally in a single computer or processor. When the study is completed, the IIIS system transitions 485 to the program & setup state 472 for the next study or part of the study. As mentioned above, some studies involve first taking a scout or other image, to, for example, allow selection of the section of the patient to be subsequently imaged. Then there can optionally be a test injection or test bolus scan to, for example, measure the propagation of the fluid through the patient's body to the area being imaged. Information from this test bolus scan can be used by the IIIS to set parameters for the imaging scan as described, for example, in PCT International Patent Application Nos. PCT/US05/41913 and PCT/US05/42891, the disclosures of which are incorporated herein by reference. Then the imaging scan is taken. In the example discussed above, the IIIS system goes through three cycles from state 472 to 476 and back again to complete the prescribed patient imaging. If the systems were cooperating according to the current art, the user would have to go back and forth between the two systems, to at least arm the injector, take the test injection, and arm the injector again for the diagnostic image. It can be appreciated from the description herein how the integrated system of this invention improves the user's situation.

Integrating the imager and injector facilitates the design of a common algorithm or algorithms that can access image data in the imager native format and control parameters of the imager aspect (for example, slice or scan thickness, imaging energy, and imaging speed) and parameters of the injector aspect (for example, flow rate and concentration). Integration also facilitates the real time assessment of the patient response function or patient model.

The state diagrams discussed herein are simplified for clarity and the level and partitioning have been chosen to assist in understanding certain aspects of several embodiments of the present invention. In all the states shown herein, there can be sub states, for example state 413 within state 412, or concurrent processes, for example reviewing images from a previous study, outside of the state that are not shown in the diagram for clarity of understanding. These and similar details are known to and/or understood by those skilled in the art.

When implementing the integrated IIIS state diagram, it can be implemented in various hardware and software known to those skilled in the art. It can, for example, be implemented in a single processor, or in multiple processors on a common bus with software that is sufficiently linked and synchronized to implement the state diagram. The current MEDRAD® STELLANT® injector system uses multiple processors within itself. The integration described herein allows the designers of the hardware and software to partition computation power as best needed to optimize their design constraints, which, for example, include a mix of speed, cost, power consumption, and reliability.

Because of the possibility of a failure of selected aspects related to just the injector or just the imager functions, it is desirable that one function be able to operate with the other. However, a potential downside of integration is the fact that more parts are common to both functions. For example, if a part of the common power conditioning apparatus failed, both the injector and imager would be without power. Similarly, parts of the single user interface could fail, and the user would not be able to operate the IIIS at all. This is an acceptable trade off in most situations, considering the benefits of integration. In those cases where it is not, redundancy or appropriate design partitioning can be employed.

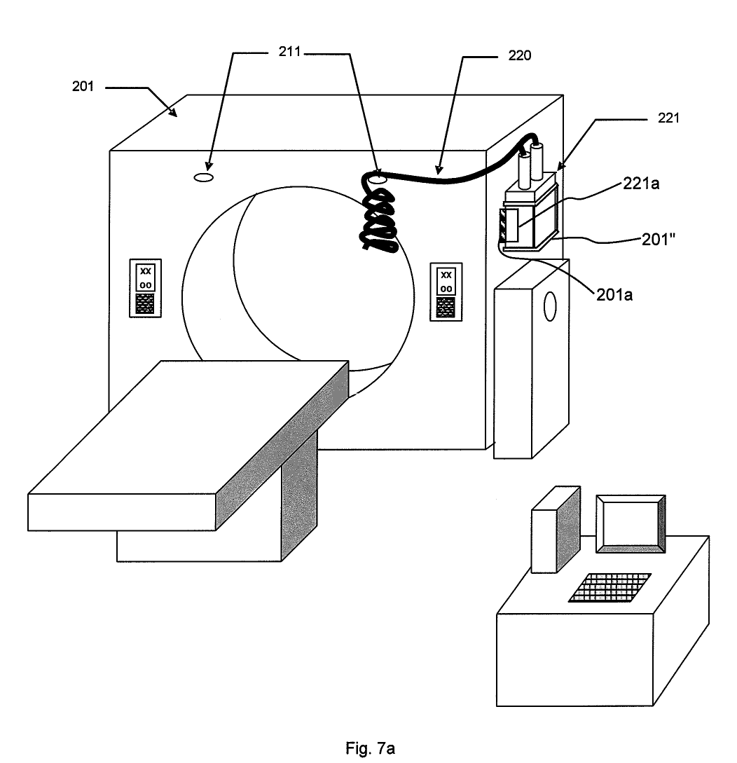

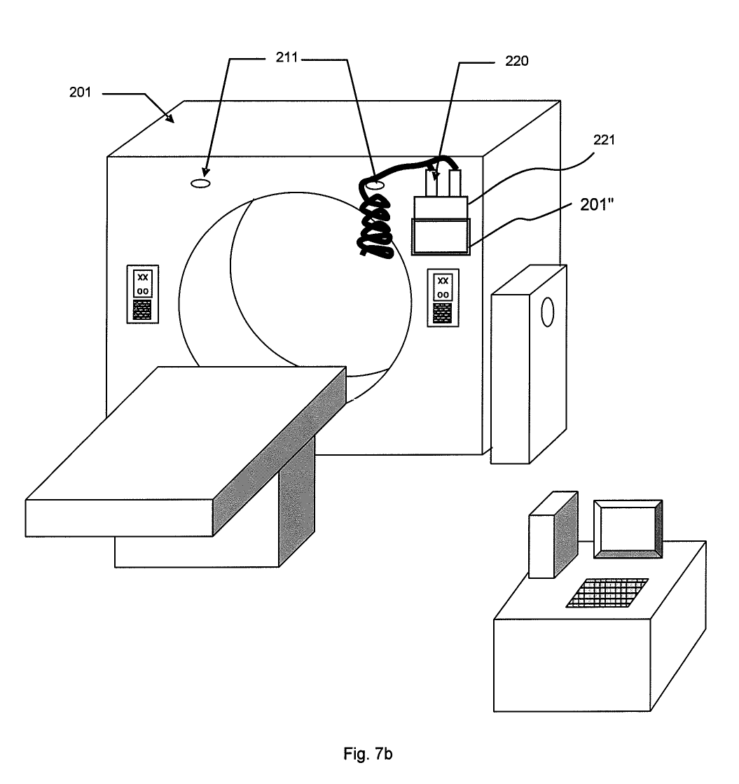

FIG. 7a shows an alternate embodiment in which the fluid injector 221 component of the IIIS is mounted on the side of housing 201′ the IIIS image acquisition apparatus 201 via for example a seating 201″. A port 221a can be provided on injector 221 to place injector 221 in operative connection with a port 201a of imager 201 to, for example, effect communications to enable integration of the control systems (via, for example, integration of state machine states) thereof as described above. The ports 221a and 201a can also enable additional integration, for example power or the fluid path. Common seatings and/or ports and interaction protocols can be used to provide for operative connection/integration of various injectors (for example, from a variety of injector manufactures) with imager 201. While potentially not perceived as “fully” integrated by a user, there are several benefits provided by the embodiment of FIG. 7a. In that regard, it is easier to rotate the fluid injector 221 to facilitate removal of air. Seating 201″ (as well as any communication port connection(s)) can, for example, be rotatable. Depending upon the details of the installation of the fluid paths, having access on three sides of the injector 221 can be beneficial. In addition, it is easier to visually check status of the fluid reservoirs from a moderate distance. FIG. 7b illustrates an embodiment of the present invention, similar to that of FIG. 7a, wherein injector 221 is mounted on the front of imager 201.

In a situation that is similar to the ability to integrate display, modem, or network cards into a personal computer, the ability to integrate injectors from various manufacturers into the IIIS has benefits of user choice and upgradeability while maintaining the benefits of integration. From the imager manufacturer's point of view, having a standardize mounting arrangement including a standardize port for communications and other integrative aspects, enables the imager manufacturer to choose among injector manufactures without any need to modify their IIIS product. Although it may seem paradoxical, design and definition of a more complete or more inclusive levels of integration can make it easier to change out pieces or subsystems, by standardizing more aspects of the injector and imager aspect interaction. The injector becomes more an integral and thus interchangeable part of the IIIS, analogous to the ability to interchange X-ray tubes from various manufacturers with identical form factors and electrical characteristics.

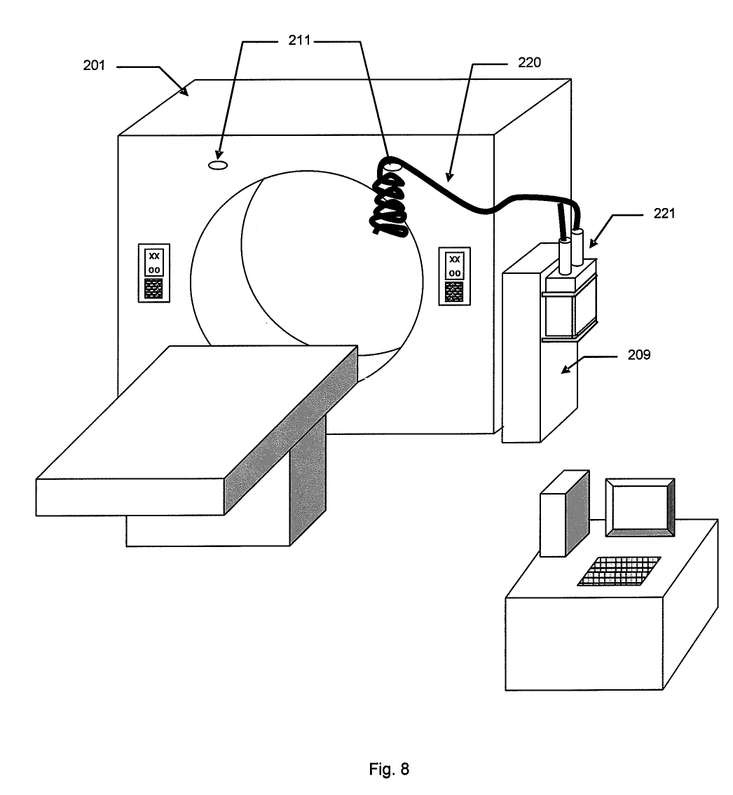

FIG. 8 shows an alternate embodiment in which the fluid injector 221 is mounted on the support or mounting post 209 of the IIIS imager 201. This embodiment has the benefit of being lower for easy user access and operation, especially by shorter users. In the case of CT, the mounting post 209 does not tilt as the IIIS imager 201 is tilted.

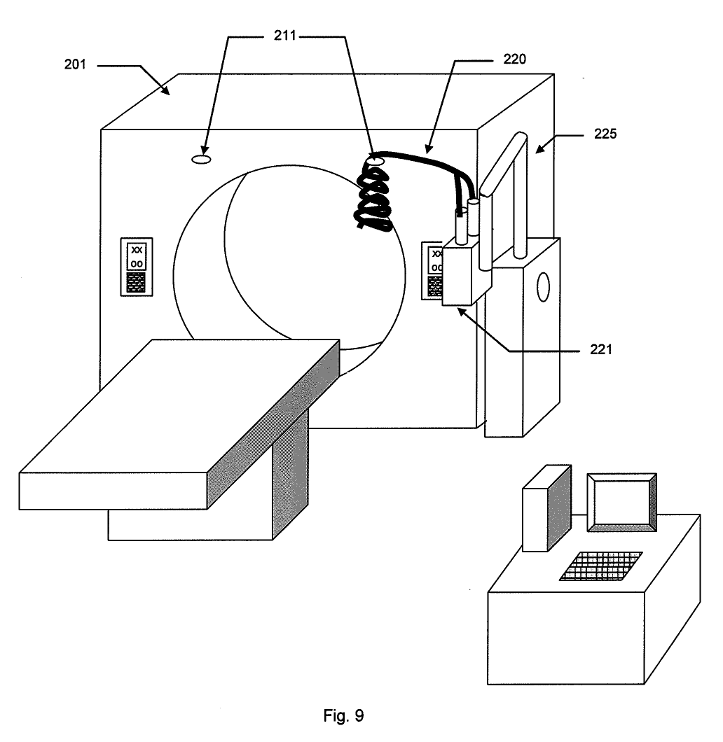

FIG. 9 shows an alternate embodiment in which the fluid injector 221 is mounted on a positionable arm or movable mount 225. This allows the fluid injector to be easily moved to either the front or the back of the imager 201. Fluid injector 221 can also be moved to occupy space above the top of imager 201 to remove it from an operator's workspace during certain portions of the injection/imaging procedure. Many similar moving mounts known, for example in the medical equipment or manufacturing tool positioning arts are practical. There can be more segments to the arm than illustrated in FIG. 9 to facilitate positioning. In some situations or room arrangements, it can be preferable that the fluid injector 221 be able to be moved up above head level to free floor space. Often in the present art, separate fluid injector heads 121 are mounted on overhead counterpoises so that they can be moved up and out of the way when not in use. Such movement can be readily accommodated in, for example, the embodiment of FIG. 9. Physical attachment or connection is beneficial but not a requirement to achieve the majority of integrative benefits of this invention. As is apparent from the descriptions and embodiments of this invention, physical connection, as used herein, is more than the provision of a communications cable or wireless communication link as could be used to achieve the injector—imager communications discussed in U.S. Pat. No. 6,970,735 B2

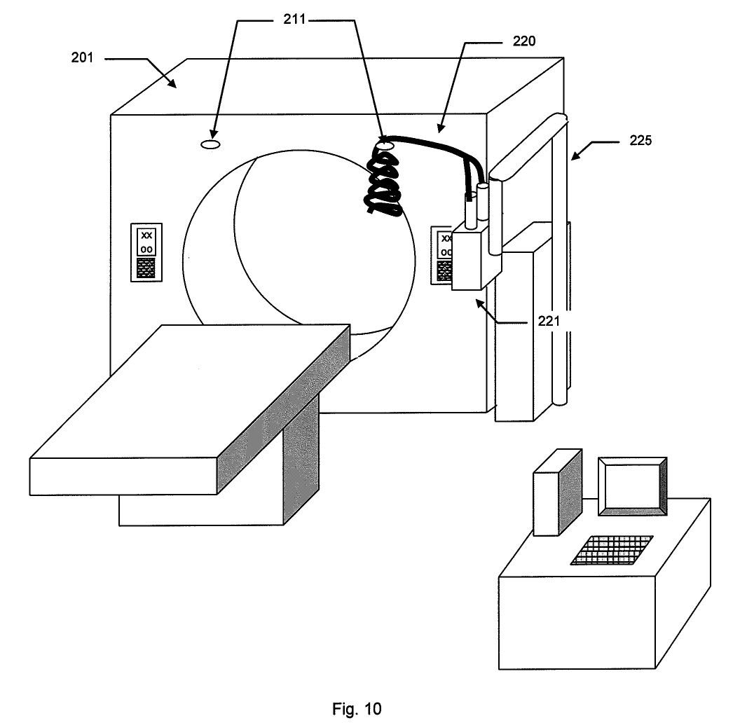

FIG. 10 illustrates an alternate embodiment in which the movable mount 225 is attached to the floor of the room rather than directly to the IIIS image acquisition apparatus 201.

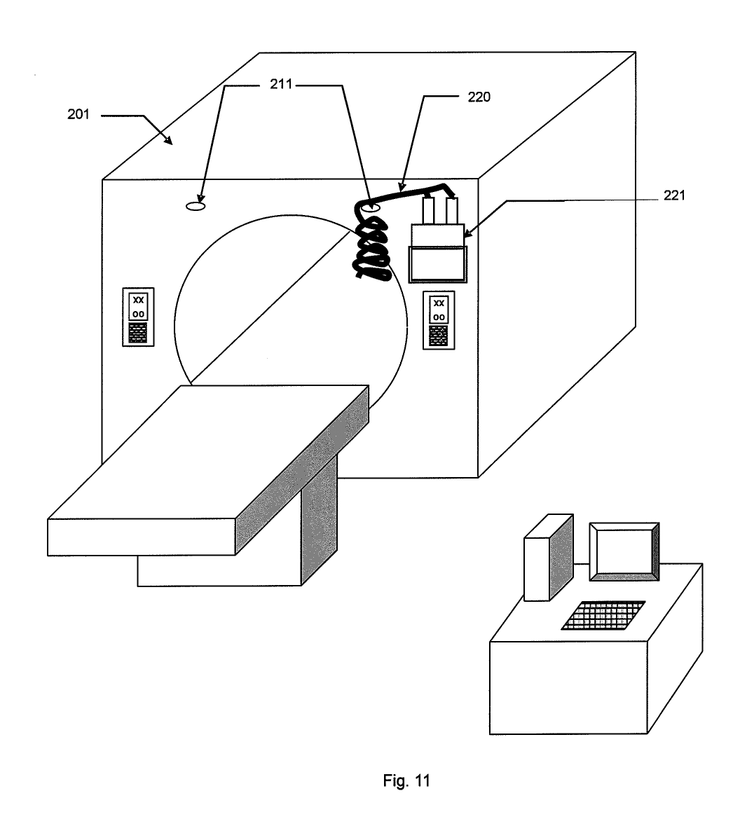

FIG. 11 illustrates an alternate embodiment in which the fluid injector 221 is mounted externally on the IIIS imager 201, in this case a horizontal field MRI imager. The majority of MRI imagers do not tilt or move. For those MR imagers that do, the mounting options discussed previously and similar ones known to those skilled in the art may be applied.

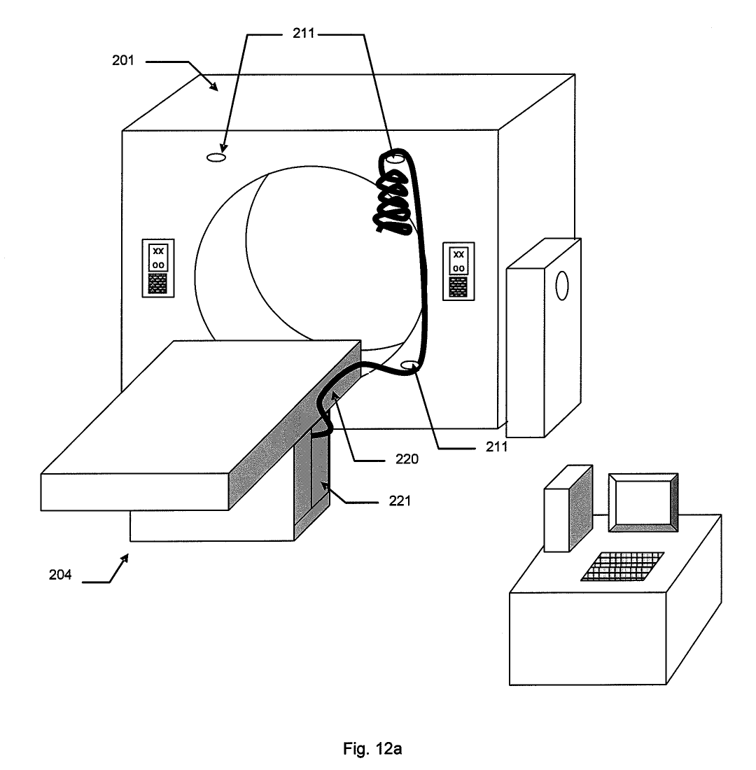

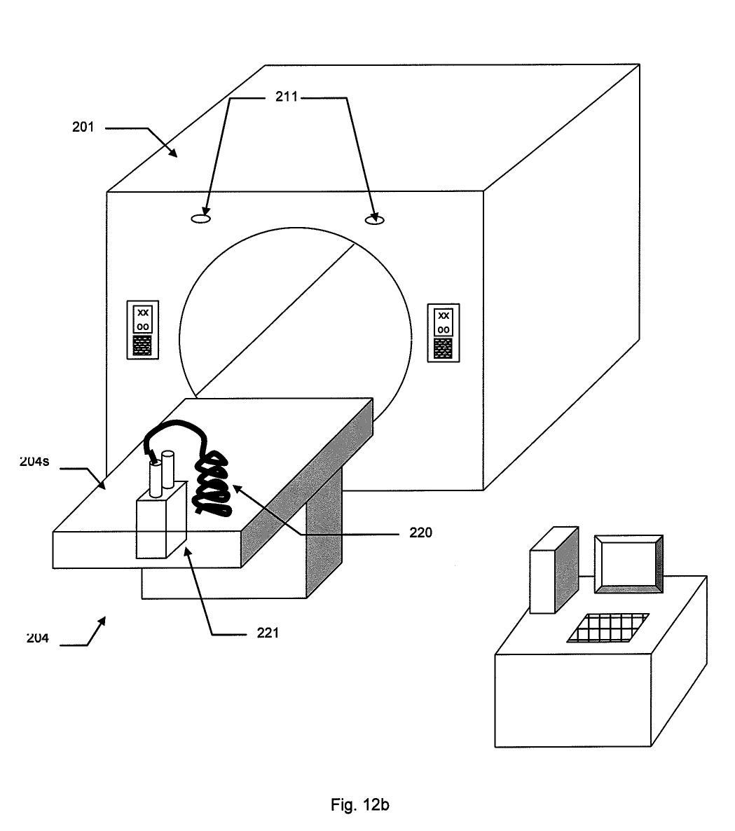

FIGS. 12a and 12b illustrate alternate embodiments in which the fluid injector 221 is mounted in association some other aspect of the IIIS In FIG. 12a, it is in association with unmoving section of the patient couch 204. In FIG. 12b it is in association with the moving section 204s of the patient couch 204. This space is generally underutilized and so is readily available. Covers for the disposable patient couch 204 can also be stored in that area as well. One drawback is that the operator may have to bend down to fill, clean, or prepare the fluid injector 221. However, if replaceable modules are used in a multipatient system, as discussed in U.S. Pat. Nos. 5,806,519, 5,885,216, 5,843,037, 6,149,627, 6,306,117, 5,739,508, 5,920,054, 5,569,181, 5,840,026, this inconvenience can be minimized. In fact, additional supplies can be stored there so they are readily available, or the system can be pulled out and pivot upwards to provide for easy access when changing fluid path segments or modules and contrast containers. A benefit of the arrangement in FIG. 12b is that, because the fluid injector 221 moves with the bed 204 and the patient, the fluid path to the patient 220 is not pulled or stretched. This is especially beneficial in MR where the patients arms are almost always at their side.

Through the physical integration in the injector and image, there are a number of benefits including saving of floor space and closeness of the injector to the patient. In addition, especially in MR, fixed positioning of any motors or magnetic material with respect to the magnetic field simplifies shielding of both the motor and the imager and shimming of any distortions. Physical integration can be used to support or improve the shielding of the IIIS. For example, in a number of the embodiments discussed above, EMI shielding can enclose both the image sensor and the injector to protect from outside interferences. Although, because of the integration, there can be internal shielding needed to protect various part of the IIIS from noise emanating from other parts of the IIIS. A second kind of shield that can be integrated in some types of imaging is ionizing radiation shielding. In this situation, the shielding prevents ionizing radiation from needlessly affecting the patient, the operator, or the correct operation of the IIIS or devices associated with the medical procedure being conducted. Although, because of the integration, there can be requirements to shield some aspects of the IIIS from ionizing radiation emanating from other aspects, for example the image sensors in a nuclear medicine imager must be shielded from any radioactivity in the injector reservoirs. when needed, and optionally preconnected for use. Physical integration can in some cases take advantage of space that is currently empty within housing 201′.

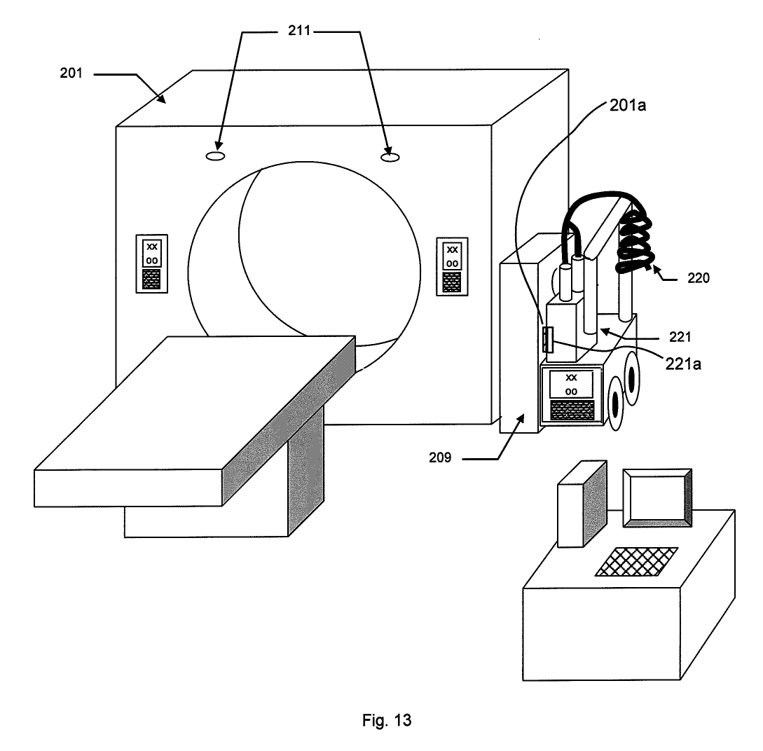

FIG. 13 illustrates an alternate embodiment in which the fluid injector 221 is mounted to the IIIS imager 201, in this case at mounting post 209. In addition, fluid injector 221 is detachable from the IIIS imager 201 so that it can be moved and be associated (for example, via seatings and standardized or adaptable communication ports as described above) with a second imager (not shown). This allows the equipment to be shared among multiple imagers. When the fluid injector 221 is attached to (or separated from) the IIIS imager 201, the software and hardware recognize its attachment and optionally modify the user interface, state machine, power conditioning, communications, programming, and other functions accordingly. Another situation where the portability of an injector aspect of the IIIS is useful is in the case of nuclear medicine imaging, where the injector had considerable radiation shielding and may be preloaded by the radiochemist in the hot lab and then be moved into the imaging suite or room for reintegration and operation in the IIIS. Example embodiments of such a system are given in U.S. application Ser. No. 60/910,810.

This integrated and detachable fluid injector 221 is also advantageous in nuclear medicine or PET imaging where the drug to be injected is radioactive. In the case of PET, the fluid injector 221 can include sufficient radioactive shielding to protect the operators, and can be moved to the drug generation equipment for filling, and then moved to the imager area for delivery.

In addition, integrated injectors for small fluid volumes, such as those for therapeutic drugs and small volumes of image contrast, such as MR or ultrasound, can be mounted or held in association with the patient, for example on their arm or on the couch 204 next to them.

The above examples of integrated imager injector systems of the present invention include an imager and a single injector for injecting, for example, contrast and saline. Alternate embodiments of integrate systems of the present invention can, for example, include an injector for other drugs, for example pharmacological stress agents, for example dobutamine or adenosine, for creating cardiac stress or beta blockers to lower heart rate. Neuroreceptor related pharmaceuticals are another example of a drug that can be injected. The integrated delivery of drug, imaging contrast, and image acquisition provide an include an injector for other drugs, for example pharmacological stress agents, for example dobutamine or adenosine, for creating cardiac stress or beta blockers to lower heart rate. Neuroreceptor related pharmaceuticals are another example of a drug that can be injected. The integrated delivery of drug, imaging contrast, and image acquisition provide an opportunity to simplify and optimize the imaging sequence to achieve the diagnostic information in the best way possible. In these examples, it is likely that heart rate and possibly blood pressure will be monitored, either through an external sensor 20, or through a physiological sensor that is part of the IIIS. Many CT and MR machines have an integrated ECG monitor because the image acquisition and or reconstruction is synchronized to the heart motion via the ECG. In this case the IIIS's integration of sensor data, fluid delivery system, and imager through a dosing and imaging algorithm enable it to maintain the patient's heart rate slow enough for optimum imaging, fast enough for patient safety, and steady enough for the completion of the procedure. And that heart rate value can affect the fluid delivery parameters and scan parameters as discussed herein. The timing of the fluid delivery and image acquisition and reconstruction can be related to the phase of the cardiac, respiratory, or other patient physiological state or cycle.

Therapeutic fluids can also be delivered via an integrated system of the present invention. Examples include, but are not limited to, drugs for thrombolysis and tumor chemotherapy. The progress of treatment could be periodically and optimally monitored and controlled. Additional examples of therapeutic fluids include anesthesia agents, nitroglycerine, heparin, cells, gene delivery agents, molecular imaging agents, and microscopic drug vesicles or bubbles that can be activated, for example by ultrasound energy.

The injector or additional system components can, for example, be added in a “plug and play mode”, conceptually similar to the addition of components to a personal computer utilizing the WINDOWS® Operating system sold by Microsoft, Inc. If a fluid delivery module is added to the IIIS, the appropriate control screen is activated on the user interface 202p and appropriate sub states are added to or activated in the IIIS Control and Coordination Unit 203. This would allow an injector or additional system components to readily be moved from one imager to another as desired or needed, for example in the case of a failure of one piece of equipment but not the other, or in the case of the purchase of a newer model of one but not the other.

In the above embodiments, the injector 100 is integrated with the imager 300 along several dimensions. The user interface is integrated so that a single interface programs and operates the integrated imager injector system 200. The functional state or system states are integrated. In the above embodiments, the devices are physically integrated to various degrees, with the various level of physical integration providing various benefits. Optionally, as shown in FIG. 2 and several of the other figures, there can be multiple user interfaces with selected subsets of control capability for the various parts, systems, or subsystems, for example the imager, injector, or monitoring appropriate for the location of the user interface and the tasks the operator needs to accomplish when in that location.

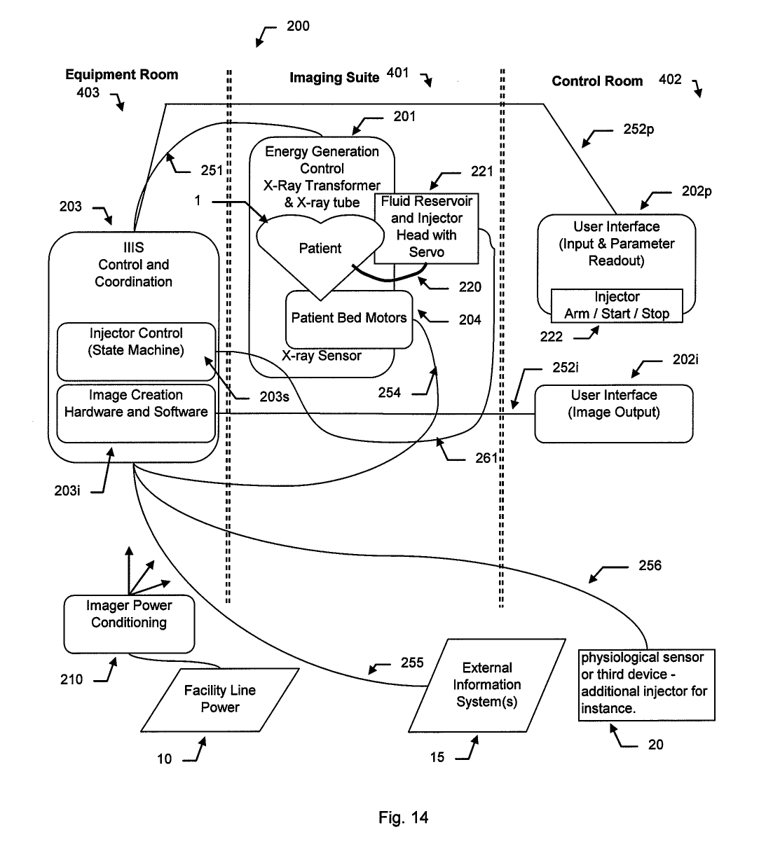

The level of functional state interaction can vary along a range, for example, from the common situation of no cooperation (or cooperative interaction via the user), to the current limit of the available products of cooperation via transmission of limited data between the injector 100 and imager 300, to the full functional state integration of this invention, as illustrated, for example, in FIG. 6. An interim step (or possibly a permanent step) is to integrate the units more than the current art, but less than the embodiment of FIG. 6. FIG. 14 illustrates an embodiment of such a partial integration. In this embodiment, there is an injector state control machine 203s that operates within the imager state control and coordination unit 203. This could be embodied as separate software that operates on the same hardware as the IIIS state machine and is tightly coupled or closely communicates with the overall IIIS state machine. Many of the real time operating systems and programming languages provide environments with this capability. Or, the injector control state machine 203s could operate on a separate piece of computer hardware that is integrated into the imager state control and coordination unit 203, preferably operating on a common bus or communications path. Lesser levels of integration may be preferred if two or more manufacturers write or build the various hardware or software components. By having less complete integration with defined interfaces, it can be easier to compartmentalize the design, building, and testing of the hardware and software, while the speed of communications can be much faster, richer, and in greater detail than in the current state of the art. This integration of state machine capability and real time or injection time control is not to be confused with the combination of the independent injector and imager data communications and user interface aspects previously disclosed in U.S. Pat. No. 6,970,735 B2 and US Published Patent Application No. 2005/0203389 A1.

These various integration levels can, for example, be viewed as forming an example path of integration evolution or growth. In a well selected embodiment, the various levels of integration beyond the current state of the art bring the user benefits, but the implementation details are generally transparent to or unnoticed by the user. In several embodiments, the more the system is perceived to be fully integrated by the user, the more the system can be perceived to be fully integrated in benefit to the user.

There is a range or continuum of levels of user interface integration as well. These separate levels of integration can be more apparent to the user. In the fully integrated embodiment, the user programs both the injector and the imager from one device, 202p, and makes no significant distinction between the imaging and fluid delivery functions. A protocol includes both imaging and fluid handling parameters in what that the user thinks of as a seamless whole. There can be recommended scan and injection sequences depending upon the region of the body to be imaged and the disease or diseases being considered. The injector portion of the user interface is only accessed if needed by the scan or the user.

However, because of the strong brand recognition and reliability of some fluid delivery device manufacturers, it may be advantageous for the IIIS user interface 202 to preserve the screen and programming sequence of the separate injector 100. This would also have the benefit of ensuring that the safety dialog would be familiar to the users. This could be readily accomplished by having the something similar to the screens of the separate injector user interface 122 appear on the IIIS user interface 202p, preferably providing injector brand recognition for the user, similar to the “Intel inside” brand campaign, if the injector and imager are manufactured by separate organizations, even if within the same company. The IIIS state machine behind the user interface could be a fully integrated embodiment such as illustrated in FIG. 6, or it could be a partially integrated embodiment such as illustrated in FIG. 14, with a separate injector control state machine 203s, or the other alternatives discussed herein. Alternatively, there could be a little less integration, in that one or more discrete injection function related keys could be included on the screen (for cursor or touch activation), on the user input device 205, or on a separate partial user interface 222. Various sophisticated user interfaces could be applied, such as wireless PDAs or heads up displays on user glasses or goggles.

In all these embodiments, there are some time critical safety functions that can be communicated from one functional block to another. In some current injectors there are hard cursor or touch activation), on the user input device 205, or on a separate partial user interface 222. Various sophisticated user interfaces could be applied, such as wireless PDAs or heads up displays on user glasses or goggles.

In all these embodiments, there are some time critical safety functions that can be communicated from one functional block to another. In some current injectors there are hard wired switches that can stop the injection immediately. In very old products, these were connected through analog circuitry to a relay that opened the current to the motor. In many modem systems, the switches are connected to a computer that periodically checks the status of the switch and acts appropriately. The response time is essentially instantaneous in human time, tens of milliseconds are sufficient. There are many digital communication protocols and constructs, including hardware and software that allow for the prioritization of messages so that high priority messages are transmitted and acted upon quickly. The lowest level example is the switch and wires to the computer input mentioned above. It is possible for multiple switches to be placed in parallel so that depressing any one switch causes the injector to stop. This is conceptually similar to the pull cords used to request that the bus driver stop at the next stop. A number of the communications protocols known to those skilled in the art, a subset of which are discussed and referenced herein have more sophisticated message prioritization methods that are sufficient. And, in a fully integrated system, the system designers and architects can specify all aspects of the hardware and software and so may design and implementation options are available. This may include a dedicated communications path 252ps (see FIG. 4a) for time and safety critical signals within path 252p or in addition to path 252p.

Physical integration provides benefits to the user, whether or not other integrations are involved. For example physically including the injector head into or onto the imager as represented in, for example, FIGS. 2, 7a, 7b, and elsewhere reduces clutter in the procedure room. This is especially appealing if different manufacturers manufacture similar injectors. Then the user could select the injector to be placed in the imager, and the injector manufacturer could provide one or more separate system components.

An imaging system includes, for example, at least an energy sensor, image creating apparatus (generally including computer hardware and software), an image display and user interface. Most imaging systems use an energy source or emitter and include a patient positioning apparatus. In imaging modalities such as nuclear medicine cameras, SPECT and PET, a radioactive element is the source of the imaging energy. Benefits of this integration, depending upon the specific energy used for image sensing and creation include reduction in amount of energy through better timing and coordination (including reduction in radiation dose to the patient), simplified data recording and communications for inclusion into the patient's records and for other uses such as procedure benchmarking, inventory control, and billing. Example embodiments related to procedure benchmarking can be found in US2003/0212707 A1, the disclosures of which are incorporated herein by reference. An integrated system facilitates closed loop control of both injection and imaging based upon patient parameters such as heart beat, breathing, and blood pressure for example. Closed loop control of selected patient parameters such as heart rate is also possible if one of the fluids injected is a physiologically active or therapeutic drug as mentioned herein. Additional potential benefits include reduced injection volumes, which can same money, as a result of tighter image feedback and more graceful handling of exceptions or problems such as extravasation. An extravasation detector (for example, as disclosed in U.S. Pat. Nos. Re38,879, RE38,695, 6,751,500, 6,487,428, 6,459,931, 6,425,878, 6,408,204, 6,375,624, 5,964,703, 5,954,668, 5,947,910, 5,334,141, and 4,877,034 as well as those disclosed in Published U.S. Patent Application Publication Nos. 2004/0215081, 2004/0176690, 2003/0004433, 2003/0036674, 2003/0036713, and 2002/0172323 and Published PCT International Patent Application Nos. WO/2003/009753, WO/2003/009752) can be part of the injector and simply stop the injection upon detection of extravasation. In several embodiments of the present invention, for example with state integration, the extravasation detector can stop both the injector and the scanner upon detection of extravasation, to, for example, minimize a radiation dose to the patient and wear and tear on the imaging system (X-ray tube for example).

A fluid injection system includes a source of one or more fluids (in one or more fluid reservoirs), a system or method to pressure the fluid for controlled flow, a fluid path from the source to the patient, and an interface for activation. Fluid reservoirs can, for example, be a syringe, a bottle, an intermediate reservoir or a container, a segment of tubing, any container holding fluid or a combination of any, some, or all of these. By integrating the injector with the imager as described herein, it is possible to overcome the problems of current spring counterbalanced overhead counterpoise units, which will rise as sufficient volume of the imaging fluid are delivered to the patient because of the decreased weight. The fluid reservoir can be integrated into the imager housing 201′.

Representative imaging modalities discussed in relation to some of the embodiments of the present invention include CT, MR, PET, SPECT, Nuclear Medicine, and ultrasound. The benefits of integration can be achieved in other imaging modalities as well. For example, integration could provide shorter contrast boluses in X-ray fluoroscopy. Infrared imaging could provide optimized dosing of fluorescent or absorbing dyes. Additional examples of imaging modalities that could benefit from integration with a drug injection system include, but are not limited to, optical imaging via endoscope, open incisions, or optical coherence tomography, impedance imaging (or impedance tomography), and thermal imaging. The imaging system acquisition apparatus can include fixed equipment, swing labs, mobile—truck mounted, mobile—wheeled for movement within a facility, and hand held imagers.

The benefits of integrating the injector and imager apply whether the injector is a single patient syringe based system such as, for example, a Mark V injector available from Medrad, Inc. of Pittsburgh, Pa. or a multipatient fluid delivery system such as described, for example, in U.S. Pat. Nos. 5,806,519, 5,885,216, 5,843,037, 6,149,627, 6,306,117, 5,739,508, 5,920,054, 5,569,181, 5,840,026.

There can be potential hurdles to be overcome by an integrated imager injector system. One hurdle is customer preference for a specific imager or injector. This has been discussed above and can be addressed by standardization of some or all of the interface between the two, for example the physical, electronic, and software interfaces. A second hurdle is regulatory approval. Currently the devices are approved separately and used by the doctor in a medical procedure. While it may appear (and may be likely) that obtaining approval of an integrated device will be more difficult, by having the devices integrated and approved as a single medical device it will be possible to implement and market algorithms and features that are not possible without the necessary integration. Regulatory or device approval integration is an example of a non-physical but none the less important integrative aspect of the present invention. In-servicing or training of technicians or operators is a second example.

Another less physical or tangible aspect of integration is business process or information related aspects, some of which have been discussed elsewhere herein. For example, an IIIS system can save or retain the aspects of the procedure performed, the contrast and disposables used, and/or other information and can transmit that information (or any portion thereof) to the manufacturer for billing and/or to purchasing or the manufacturer for restocking of the supplies used. It can also save or retain all the procedure details and communicate those details (or any portion thereof) to the patient's records or a data base for, for example, benchmarking.

Because there is likely to be a transition period from when a fully integrated IIIS is available and the universal adoption of IIISs, the present invention includes the ability to take the selected injector aspects of the IIIS, place them in a separate housing with a similar physical structure and port as provided by the imager housing 201′, add necessary hardware (such as for power conditioning and user interface), and add necessary software and processing to enable the injector to be operated independently of the IIIS. If an IIIS fails in some aspect other than those solely related to the injector, the injector could be pulled from the IIIS, placed into the separate housing and used as an independent injector with an independent imager.

The user or operator interacts with the equipment to prepare or execute a scan. Additional terms or titles for a person performing this function include doctor, technician, nurse, physiologist, and clinical engineer.

Because the integration described in the present invention can proceed along a number of somewhat or totally independent aspects or dimensions, with different levels or extents to the integration implemented in a particular embodiment, the term “cooperation” is used herein to describe the limited interactions between independent injector and imagers that exists in the current state of the art, and the term “integration” is used herein to describe embodiments of the present invention which include aspects and features as described herein that provide the benefits also described herein.

The foregoing description and accompanying drawings set forth the preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. An image acquisition system operable to obtain an image of at least a portion of a body, comprising:
an imaging system comprising at least one energy sensor to measure energy from the body, an image creating system adapted to create an image based at least in part from a signal from the at least one energy sensor, an image display in operative connection with the image creating system and a user interface in operative connection with the image creating system; and
a fluid injector system comprising at least one source of a first fluid, a pressurizing system in operative connection with the source of the first fluid, and a user interface in operative connection with the pressurizing system;
a communication system placing the imaging system and the fluid injector system in communicative connection;
the imaging system and the injector system being further operatively integrated in addition to the communication system.

2. The image acquisition system of claim 1 wherein the imaging system further comprises at least one energy source to transmit energy through the body.

3. The image acquisition system of claim 1 wherein at least one component of the imaging system is physically connected to at least one component the injector system.

4. The image acquisition system of claim 3 wherein the at least a portion of the injector system is housed within a housing of the imaging system.

5. The image acquisition system of claim 3 wherein at least a portion of a housing of the injector is attached to the imaging system.

6. The image acquisition system of claim 5 wherein a portion of the injector housing is attached to a support for the image acquisition system.

7. The image acquisition system of claim 5 wherein at least one fluid path in operative connection with the injection system is in operative connection with the imaging system.

8. The image acquisition system of claim 3 wherein at least one fluid reservoir in operative connection with the injection system is in operative connection with the imaging system.

9. The image acquisition system of claim 3 wherein at least one fluid heating system in operative connection with a fluid path of the injection system is in operative connection with the imaging system.

10. The image acquisition system of claim 3 wherein the imaging system includes at least one supply compartment adapted to house supplies for the injector system.

11. The image acquisition system of claim 1 wherein the imaging system and the injector system are in electrical connection with a common power conditioning system.

12. The image acquisition system of claim 1 wherein the imaging system and the injector system are adapted to receive common data from at least one patient physiological sensor.

13. The image acquisition system of claim 12 wherein the patient physiological sensor is an ECG sensor, a respiration sensor, a blood oxygen sensor, or a blood pressure sensor.

14. The image acquisition system of claim 1 wherein at least a portion of the control software for the imaging system and the injector system is integrated.

15. The image acquisition system of claim 14 wherein the imaging system and the injector system share at least one common state machine state.

16. The image acquisition system of claim 14 wherein the imaging system and the injector system integrate imaging parameter and fluid delivery protocols.

17. The image acquisition system of claim 14 wherein the imaging system and the injector system integrate user preferences.

18. The image acquisition system of claim 14 wherein the imaging system and the injector system share patient information.

19. The image acquisition system of claim 14 wherein the imaging system and the injector system share usage data.

20. The image acquisition system of claim 1 wherein the imaging system and the injector system share at least one common computer processor.

21. The image acquisition system of claim 1 wherein the imaging system and the injector system share at least one common computer memory.

22. The image acquisition system of claim 1 wherein the imaging system and the injector system share at least one common data communication bus.

23. The image acquisition system of claim 1 wherein the imaging system and the injector system share at least one common computer hardware component.

24. The image acquisition system of claim 1 wherein the imaging system and the injector system share at least one common safety check system.

25. The image acquisition system of claim 1 wherein the imaging system and the injector system share at least one common user interface.

26. The image acquisition system of claim 1 wherein the imaging system and the injector system share at least one common display.

27. The image acquisition system of claim 1 wherein the imaging system and the injector system share at least one common communication port to at least one other information system.

28. The image acquisition system of claim 27 wherein the information system is a hospital information system.

29. An image acquisition system operable to obtain an image of at least a portion of a body, comprising:
an imaging system comprising at least one energy sensor to measure energy from the body, an image creating system adapted to create an image based at least in part from a signal from the at least one energy sensor, an image display in operative connection with the image creating system and a user interface in operative connection with the image creating system; and
a fluid injector system comprising at least one source of a first fluid, a pressurizing system in operative connection with the source of the first fluid, and a user interface in operative connection with the pressurizing system;
the imaging system and the injector system being operatively integrated in at least one aspect other than communication of data between the imaging system and the injector system.

30. An image acquisition system operable to obtain an image of at least a portion of a body, comprising:
an image creating system adapted to create an image based at least in part from a signal from the at least one energy sensor, an image display in operative connection with the image creating system and a user interface in operative connection with the image creating system; and
a fluid injector system comprising at least one source of a first fluid, a pressurizing system in operative connection with the source of the first fluid, and a user interface in operative connection with the pressurizing system;
the imaging system and the injector system being operatively integrated in at least two of the following aspects: physical connection, data input via at least one common user interface, displaying of information via at least one common display, electrical connection to at least one common power conditioning system, receipt of common data from at least one patient physiological sensor; at least one common communication port to at least one information system, and a common control system.

31. The image acquisition system of claim 30 wherein the at least a portion of the injector system is housed within a housing of the imaging system.

32. The image acquisition system of claim 30 wherein at least a portion of a housing of the injector is attached to the imaging system.

33. The image acquisition system of claim 32 wherein a portion of the injector housing is attached to a support for the image acquisition system.

34. The image acquisition system of claim 30 wherein at least one fluid path in operative connection with the injection system is in operative connection with the imaging system.

35. The image acquisition system of claim 30 wherein at least one fluid reservoir in operative connection with the injection system is in operative connection with the imaging system.

36. The image acquisition system of claim 30 wherein at least one fluid heating system in operative connection with a fluid path of the injection system is in operative connection with the imaging system.

37. The image acquisition system of claim 30 wherein the imaging system includes at least one supply compartment adapted to house supplies for the injector system.

38. The image acquisition system of claim 30 wherein at least a portion of the control software for the imaging system and the injector system is integrated.

39. The image acquisition system of claim 38 wherein the imaging system and the injector system share at least one common state machine state.

40. The image acquisition system of claim 38 wherein the imaging system and the injector system integrate imaging parameter and fluid delivery protocols.

41. The image acquisition system of claim 38 wherein the imaging system and the injector system integrate user preferences.

42. The image acquisition system of claim 38 wherein the imaging system and the injector system share patient information.

43. The image acquisition system of claim 38 wherein the imaging system and the injector system usage data.

44. The image acquisition system of claim 30 wherein the imaging system and the injector system share at least one common computer processor.

45. The image acquisition system of claim 30 wherein the imaging system and the injector system share at least one common computer memory.

46. The image acquisition system of claim 30 wherein the imaging system and the injector system share at least one common data communication bus.

47. The image acquisition system of claim 30 wherein the imaging system and the injector system share at least one common computer hardware component.

48. The image acquisition system of claim 30 wherein the imaging system and the injector system share at least one common safety check system. at least one common communication port to at least one other information system.

49. An imaging system comprising at least one energy sensor to measure energy from a signal from the at least one energy sensor, an image display in operative connection with the image creating system and a user interface in operative connection with the image creating system; the imaging system being adapted to be integrated with a fluid injector system in at least two of the following aspects: physical connection, user interface, electrical connection to at least one common power conditioning system, receipt of common data from at least one patient physiological sensor; at least one common communication port to at least one information system, and a common control system.

50. A fluid injector system comprising at least one source of a first fluid, a pressurizing system in operative connection with the source of the first fluid, and a user interface in operative connection with the pressurizing system; the injector system being adapted to be integrated with an imaging system in at least two of the following aspects:. physical connection, user interface, electrical connection to at least one common power conditioning system, receipt of common data from at least one patient physiological sensor; at least one common communication port to at least one information system, and a common control system.

51. The imaging system of claim 49 wherein said imaging system is adapted to integrated with a fluid injector system in at least four of the said aspects.

52. The fluid injector system of claim 50 wherein said fluid injector system is adapted to integrated with an imager system in at least four of the said aspects.
